(19) United States
(12) Patent Application Publication
(10) Pub. No.: US 2003/0216678 A1
(43) Pub. Date: Nov. 20, 2003

(54) Methods for inducing vascular remodeling and related methods for treating diseased vascular structures

(76) Inventors: Keith March, Carmel, IN (US); Richard Popp, Los Altos Hills, CA (US); Peter Fitzgerald, Portola Valley, CA (US); Patrick Cahalan, Nashua, NH (US); Daniel Burkhoff, Tenafly, NJ (US); Laurence Roth, Windham, NH (US); Nancy Briefs, Nashua, NH (US); William Santamore, Medford, NJ (US); Robert Swain, Bedford, NH (US); Peter Boekstegers, Diessend A, DE (US); Vincent Pompili, Chagrin Falls, OH (US)

Correspondence Address: Finnegan, Henderson, Farabow,, Garrett & Dunner, L.L.P., 1300 I Street, N.W., Washington, DC, 20005-3315, US

(21) Appl. No.: 10/388,592

(22) Filed: Mar. 17, 2003

Related U.S. Application Data
(60) Provisional application No. 60/364,632, filed on Mar. 18, 2002.

(51) Int. Cl. A61F 2/04

(52) U.S. Cl. 604/8; 623/904

(57) ABSTRACT

A method for inducing vascular remodeling and related methods for treating patients having diseased vasculature. The method of inducing remodeling of the vascular system includes altering the blood flow dynamics in portions of the vascular system via mechanical mechanisms. Remodeling of certain coronary vasculature may be induced by creating a passage in a heart wall between a heart chamber and a blood vessel so as to permit blood flow communication, for example, direct blood flow communication, between the chamber and the vessel. A method of inducing vascular remodeling may include providing a blood flow passage between a heart chamber and the coronary vessel, or other flow dynamics altering structure, such that enlargement of at least a portion of the coronary vessel occurs.

This application claims priority to U.S. Provisional Application No. 60/364,632, filed Mar. 18, 2002, the entire disclosure of which is incorporated by reference herein.

### BACKGROUND OF THE INVENTION

1. Field of the Invention

The present invention relates to a method for inducing vascular remodeling and related methods for treating patients having diseased vasculature. In particular, the invention relates to inducing remodeling of the vascular system by altering the blood flow dynamics in portions of the vascular system via mechanical mechanisms. For example, remodeling of certain coronary vasculature may be induced by creating a passage in a heart wall between a heart chamber and a blood vessel so as to permit direct blood flow communication between the chamber and the vessel.

2. Description of the Prior Art

Attempts to revascularize the heart are well known. For example, angiogenic drugs may be delivered to ischemic portions of a heart wall to induce the formation of capillaries within the heart wall. It has been thought that such capillary formation would increase blood flow to the heart wall to revive the ischemic tissue and improve the heart's pumping efficiency. However, studies suggest that achieving improved revascularization results, i.e., a significant improvement in the flow to the distal bed, may be accomplished through arteriogenesis, or the growth of relatively small collaterals, such as arterioles, to larger conductive collaterals. Moreover, in certain people, physical exertion, such as exercising, for example, may cause a naturally-induced vascular remodeling response, such as, for example, arteriogenesis and an increase in the diameter of certain coronary vessels. Such vascular remodeling may lead to improved blood flow through the vascular system, which in turn may prevent, hinder, or lessen the negative effects of diseases associated with the vascular system, including various coronary diseases. Further, such vascular remodeling may improve the overall efficiency of various functions performed by the vascular system.

Vascular remodeling due to exercise, however, is relatively difficult to control or predict and often people having certain diseased vascular systems are not able to exercise comfortably. Due to the relatively limited options currently available for achieving remodeling of the vascular structure, it may be desirable to provide another technique that induces remodeling of the vascular structure, including in people for whom exercise does not present an appropriate option. Moreover, it would be desirable to provide a technique capable of achieving significant vascular remodeling results, over and above that observed in other settings. For example, a technique that significantly enlarges a diseased vascular structure and/or other native vascular structures to improve blood flow and/or that significantly increases the growth and number of collateral vessels to the diseased structure and/or other native structures would be desirable.

Diseases affecting the vascular system, including those mentioned above, and in particular the coronary vascular system, are a major problem throughout the world. A relatively common disease affecting coronary arteries, as well as other blood vessels, includes occlusion of the vessel with plaque or other tissue due to the formation of stenotic lesions on interior walls of the vessel. For purposes of this disclosure, an “occluded,” “diseased,” or “stenosed” vessel includes vessels that are either partially occluded by, for example, one or more stenotic lesions that permit some blood to flow through the stenosed region of the vessel, and vessels that are completely occluded by, for example, a stenotic lesion that substantially or completely blocks the flow of blood through the stenosed region of the vessel.

At the very least, an occluded coronary vessel may impair the efficiency of the heart's pumping action. Even worse, an occluded vessel can lead to heart attack and/or death. In some cases, such occluded arteries and/or other vessels may be treated through less invasive percutaneous techniques such as balloon angioplasty, intracoronary stenting, vessel-to-vessel bypass, and atherectomy. A common treatment for occluded vessels, and occluded coronary arteries in particular, is coronary artery bypass grafting (CABG). CABG typically involves inserting one or more vessel grafts (e.g., venous arterial segments) between the aorta and the coronary artery. The inserted graft delivers blood from the native arterial graft (i.e., internal mammary artery) or from a venous graft connecting the aorta to a point downstream of the occlusion in the artery, thereby bypassing the blocked portion of the artery. For purposes of this disclosure, such conventional techniques, including those specifically mentioned, will be referred to collectively as “conventional coronary vessel procedures.”

Conventional coronary vessel procedures may not be suitable for certain patients for a variety of reasons. For example, CABG procedures may not be suitable for diabetic patients who often display constricted and relatively rigid coronary vessels. The vessels of such patients often are too narrow and/or noncompliant (i.e., rigid) to insert the bypass graft segment. Conventional coronary vessel procedures also may not be suitable for patients who have diffusely diseased coronary vessels. In these patients, several stenotic lesions may develop along a length of the coronary vessel wall. Treating such numerous lesions via intracoronary stenting, balloon angioplasty, bypass grafting, or atherectomy would not be practical, even if it were possible.

Further, even if conventional coronary vessel procedures can be performed on a patient, in certain patients the distal bed (i.e., the arterioles, capillaries, venules, and other vascular structures that transport blood from the coronary artery to the heart tissue) has a relatively high resistance such that it substantially restricts perfusion. When this condition is present, conventional coronary vessel procedures are likely to be unsuccessful, since the ultimate goal of these procedures is to replenish the perfusion of the heart tissue.

In addition, even when certain conventional coronary vessel procedures are suitable for patients, those procedures can have drawbacks. For example, the patency of bypass grafts may be relatively short-lived. Often vein grafts become occluded and unable to provide sufficient blood flow over time. The effectiveness of balloon angioplasty also may be short-lived, resulting in restenosis of the treated vessel.

Due to the limitations in the conventional coronary vessel procedures, it is desirable to provide an improved treatment for patients having diseased vascular systems, including patients having occluded and/or diseased (e.g., injured) coronary vessels and/or diseased, or otherwise not properly functioning, distal beds. It is especially desirable to provide a treatment capable of being implemented on various patient types including those described above. It also is desirable to provide a treatment that permits patients previously untreatable through conventional coronary vessel procedures to become candidates for those procedures. Further, it is desirable to provide a treatment for use in conjunction with conventional coronary vessel procedures that improves the patency of the vessel after those procedures.

### SUMMARY OF THE INVENTION

A relatively recent technique for treating occluded vessels includes placing an implant, for example, a conduit, in the heart wall between a heart chamber, for example the left ventricle, and a coronary vessel, for example an occluded coronary artery. Placement of the implant permits blood to flow from the left ventricle to a point in the coronary artery downstream of an occlusion. Such a technique may be used on all patient types, including the types of patients discussed above.

The inventors have found that providing a passage in a heart wall between a heart chamber and a coronary vessel or between two vessels (e.g., between a vein and an artery) may result in significant remodeling of the vascular system. For example, it has been observed that providing a passage, such as via an implant, which may include, for example, a conduit, stent, or other suitable mechanical device, or via dilation, ablation, drilling, or the like, may cause a host response that in turn may result in production of endogenous factors that can cause various biological changes. These biological changes include, for example, significant vascular remodeling of the native vessels and other collateral structures. This vascular remodeling may include, for example, the enlargement of native vessels, the growth of collateral vessels, and a reduction in ischemia. The various vascular remodeling effects that have been observed are at least in part due to alteration of blood flow dynamics created by the passage. Such altered blood flow dynamics may occur, for example, in the vascular structures being connected via the passage, and may take the form of, for example, a change in direction of normal blood flow patterns, including oscillation and frequency, increased blood flow through a structure, and increased shear stresses on vessel walls or otherwise altered shear stresses on vessel walls, for example, due to flow oscillations. Collectively, the various remodeling effects observed by the inventors are referred to in this disclosure as “flow-dynamics-induced vascular remodeling.”

As will be explained in detail below, the discovered flow-dynamics-induced vascular remodeling, for example, in connection with the placement of an implant in the heart wall that permits blood to flow between the left ventricle and an occluded coronary artery, not only is unexpected but also has resulted in significant biological changes, as measured in terms of both qualitative changes and quantitative changes. The flow-dynamics-induced vascular remodeling also is significantly greater than vascular remodeling effects observed in other settings. The discovered flow-dynamics-induced vascular remodeling includes, for example, an enlargement of the native vessels, such as epicardial vessels, for example, an occluded vessel, and branching intramyocardial vasculature, and the accelerated growth of both epicardial and intramyocardial collateral vessels, which may include the formation of conductive collateral vessels (i.e., arteriogenesis). Such remodeling tends to greatly improve flow through the native vessel as well as through the collateral vessels and can have a marked effect upon the overall vascular system pressures, shear stresses, and flow patterns. This in turn may counteract the reduction in the heart's pumping efficiency due to ischemia. It also may prevent, or at least hinder, the further progression of the ischemia and other cardiovascular disease processes, and may improve the perfusion of the heart's distal bed.

In addition, the flow-dynamics-induced vascular remodeling, and in particular the significant enlargement of the target vessel, for example an occluded coronary artery, to unexpected levels may permit conventional coronary vessel procedures to be implemented in patients that previously were not candidates for such treatments. For example, significant enlargement of the diameter of a relatively constricted, noncompliant vessel may facilitate anastomosis of a bypass graft to the vessel. This facilitation may occur by providing a surgeon with more flexibility in selecting anastomosis sites and/or by facilitating the actual anastomosis procedure by providing a larger, more flexible area of the vessel to work on. Without the formation of the passage in the heart wall between the left ventricle and coronary artery and the consequent vascular remodeling, performing a bypass procedure on such a patient might prove relatively difficult, if not impossible.

The flow-dynamics-induced vascular remodeling discovered by the inventors also may contribute to the improvement of so-called “distal runoff” (i.e., perfusion of the distal bed) that occurs due to the increase in diameter of distal collateral vessels, such as by the promotion of conductive collateral vessels (i.e., arteriogenesis), for example, and/or by the growth of new collateral vessels. Further, this remodeling may help improve the patency and/or successfulness of bypass grafts and other conventional coronary vessel procedures. For a conventional balloon angioplasty procedure, the observed vascular remodeling may tend to counteract negative remodeling effects, such as restenosis, associated with that procedure. This negative remodeling may be counteracted by the positive remodeling induced by the flow dynamics associated with connecting the ventricle to the coronary artery through the heart wall and permitting direct blood flow between the two.

Another setting in which a type of negative remodeling may be counteracted by the positive remodeling associated with the flow-dynamics induced vascular remodeling described herein includes post-cardiac transplant vasculopathy. In this setting, after the implantation of the donor heart in a transplant patient, late narrowing and loss of branches of the coronary tree may be due to a reduction in the size of the vessels. These conditions may be caused by the shrinkage of external elastic lamina. To combat these effects, a passage, for example in the form of a stent or other implant, may be placed in the transplanted heart, for example in the heart wall between the left ventricle and coronary artery, after transplantation to induce positive vascular remodeling. Such flow-dynamics-induced vascular remodeling may counteract the tendency of the vessels to shrink. This, in turn, may enhance the long term performance of the transplanted heart..

The flow-dynamics-induced vascular remodeling techniques described herein also may be used in patients who have previously been designated as candidates for angiogenic therapies. In such patients, the flow-dynamics-induced vascular remodeling techniques may be used alone or in conjunction with other conventional revascularization treatments.

This invention could be practiced without performing one or more of the aspects described above. Other aspects will become apparent from the detailed description that follows. As embodied and broadly described herein, the invention includes a method of treating a diseased coronary vessel comprising providing a blood flow passage between a heart chamber and the coronary vessel such that enlargement of at least a portion of the coronary vessel occurs. The method further includes selecting a further therapy to treat the diseased vessel based on the enlargement of the vessel.

In another exemplary aspect of the invention, a method of treating a coronary vessel having an occlusion comprises implanting a device in a heart wall so as to cause blood to flow through the device into the vessel at a point proximal to the occlusion.

According to yet another exemplary aspect, the invention includes a method of treating a heart which may comprise placing an implant having a passage in a heart wall between a heart chamber and a coronary vessel so as to permit blood flow through the passage between the chamber and the coronary vessel during at least a portion of a cardiac cycle and induce vascular remodeling. The method may further comprise, after a predetermined time period, ceasing to permit the blood flow through the passage throughout the entire cardiac cycle. The predetermined time period may be, for example, a time period at least sufficient to achieve the vascular remodeling.

In yet another exemplary aspect, the invention may include a method of treating a heart comprising providing a blood flow passage between a heart chamber and a coronary vessel so as to permit blood to flow between the heart chamber and the coronary vessel and induce vascular remodeling. The method further includes delivering at least one substance chosen from pharmacologic agents, drugs, and genes proximate the blood flow passage to accelerate the vascular remodeling.

According to another exemplary aspect, a method of treating a heart having an injured coronary vessel may comprise providing a blood flow passage between a heart chamber and an injured coronary vessel so as to permit blood to flow between the heart chamber and the injured coronary vessel. Thus providing the blood flow passage may induce vascular remodeling of the heart.

Yet another exemplary aspect of the invention may include a method of treating a heart comprising providing a blood flow passage between a heart chamber and a coronary vessel so as to permit blood to flow between the heart chamber and the coronary vessel to induce vascular remodeling. The method may further comprise providing a further vascular therapy for treating the heart after the providing of the blood flow passage.

Another exemplary aspect of the invention may include a method of performing a heart transplant. The method may comprise providing a blood flow passage between a heart chamber and a coronary vessel of a transplanted heart following the heart transplant. The method may further include performing the heart transplant by implanting the donor heart.

### BRIEF DESCRIPTION OF THE DRAWINGS

Besides the broad, general details and arrangements set forth above, the invention could include a number of other arrangements, such as those explained in the remainder of this disclosure. Both the foregoing summary description and the following more detailed description are exemplary. The accompanying drawings are included to provide a further understanding and are incorporated in and constitute a part of this specification. The drawings illustrate exemplary embodiments and, together with the description, serve to explain certain principles. In the drawings,

FIG. 1 is a cross-sectional view of an exemplary embodiment of a ventricular implant in a heart wall between a left ventricle and a coronary artery according to an exemplary method for treating an occluded coronary artery;

FIG. 2a is a graph showing the diameter of the left anterior descending artery (LAD) in ten pigs measured before and after placement of an implant in the heart wall between the left ventricle and the left anterior descending artery;

FIG. 2b is a graph showing the area of the proximal left anterior descending artery corresponding to the data for the ten pigs of FIG. 2a;

FIG. 3a is a graph showing the diameter of the circumflex artery (LCx) measured at a relatively proximal location in the ten pigs of FIG. 2a measured before and after placement of an implant in the heart wall between the left ventricle and the left anterior descending artery;

FIG. 3b is a graph showing the area of the circumflex artery corresponding to the data for the ten pigs of FIG. 3a;

FIG. 4a shows a cross-sectional view of the circumflex artery of a pig in which an implant has been placed according to an exemplary embodiment of the invention;

FIGS. 4b and 4c show cross-sectional views of the left anterior descending artery in a pig at two places along the vessel after remodeling of the artery has occurred due to placement of an implant according to an exemplary embodiment of the invention;

FIG. 5 is a partial cross-sectional view of the heart with an implant connecting the left ventricle and coronary artery according to an exemplary embodiment of the invention;

FIG. 6 is a partial cross-sectional view of the heart with an implant connecting the left ventricle and the coronary artery according to yet another exemplary embodiment of the invention;

FIG. 7 is a graph of average baseline coronary flow and average coronary flow with an implant in the heart wall measured at a location distal to an occluded ameroid in a canine LAD approximately 30 days after implantation of the occluded ameroid according to an exemplary embodiment of the invention;

FIG. 8 is a graph showing experimental results of how a ventricular implant affects blood flow in a coronary artery with a high grade stenosis according to an exemplary embodiment of the invention; and

FIG. 9 is a graph showing experimental results of the diameter of normal and balloon-injured vessels before and after the implantation of the ventricular implant according to an exemplary aspect of the invention.

### DETAILED DESCRIPTION OF EXEMPLARY EMBODIMENTS

Reference will now be made in detail to exemplary embodiments of the invention, examples of which are illustrated in the accompanying drawings. Wherever possible, the same alphanumeric reference labels are used in the drawings and the description to refer to the same or like structures.

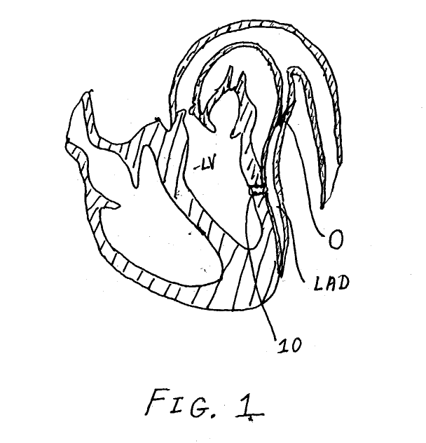

As discussed above, a relatively recent technique for treating occluded vasculature, such as a coronary artery, includes placing an implant in the heart wall so as to provide direct blood flow communication between a heart chamber, such as the left ventricle, and a coronary vessel, such as the left anterior descending artery (LAD), at a point in the LAD distal to an occlusion O, as shown in FIG. 1.

In an effort to further refine and improve this treatment technique, the present inventors conducted a study in which such an implant was placed in a heart wall of a pig between the left ventricle and an unoccluded LAD. The pig survived the implant procedure and the diameters of the LAD and the LCX were measured at 30 and 90 days via angiography.

From this study, it was discovered that the LAD experienced a significant, unexpected enlargement in diameter measured proximal to the ventricular implant over a time period as short as thirty days. In particular, prior to placement of the implant, the proximal LAD had a diameter of approximately 3.1 mm. Thirty days after placing the implant in the heart wall to connect the left ventricle and the LAD, the diameter of the LAD had increased to approximately 5.3 mm. Thus, the diameter of the artery increased by approximately 71% at 30 days. After the implant had been in the heart wall for 90 days, the diameter of the artery was approximately 7.8 mm, representing an increase of approximately 152% from its initial size.

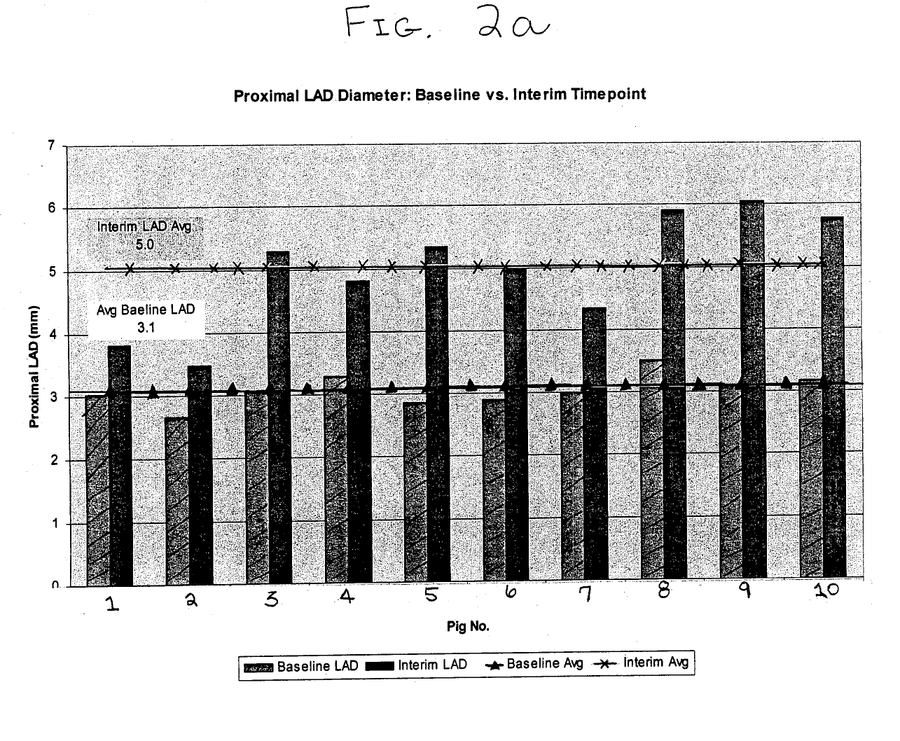

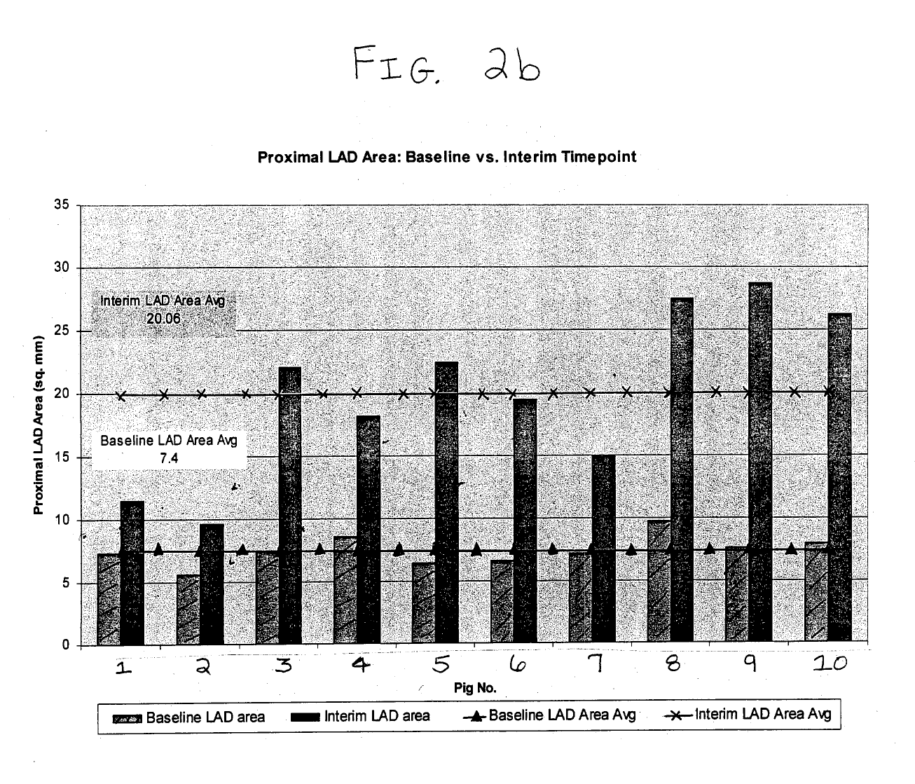

FIG. 2a shows a graph containing data regarding the proximal LAD diameter. The data shown are a measure of the diameter over a period of time in a group of ten pigs having an implant in the heart wall so as to provide blood flow between the left ventricle and an unoccluded coronary artery. As shown in FIG. 2a, the average baseline (i.e., prior to placement of the implant in the heart wall) diameter proximal to the implant was 3.1 mm. The interim average diameter of the LAD, measured on an average of approximately 26.5 days after having placed the implant in the heart wall, was 5.0 mm. This represents an average diameter enlargement of approximately 61% after an implant has been placed for an average of approximately 26.5 days in the heart wall between the left ventricle and the proximal LAD.

FIG. 2b shows a graph containing data regarding the proximal LAD area over time for the same group of ten pigs, and their resultant data, as set forth in FIG. 2a. As shown in FIG. 2b, the average baseline area (i.e., prior to placement of the implant in the heart wall) of the LAD, measured at the same location in the artery as the diameter measurement in FIG. 2a, was 7.4 sq. mm. The interim average area of the LAD, measured an average of approximately 26.5 days after having placed the implant in the heart wall was 20.06 sq. mm. This represents an average area enlargement of approximately 171% after the implant has been in the heart wall for an average of approximately 26.5 days.

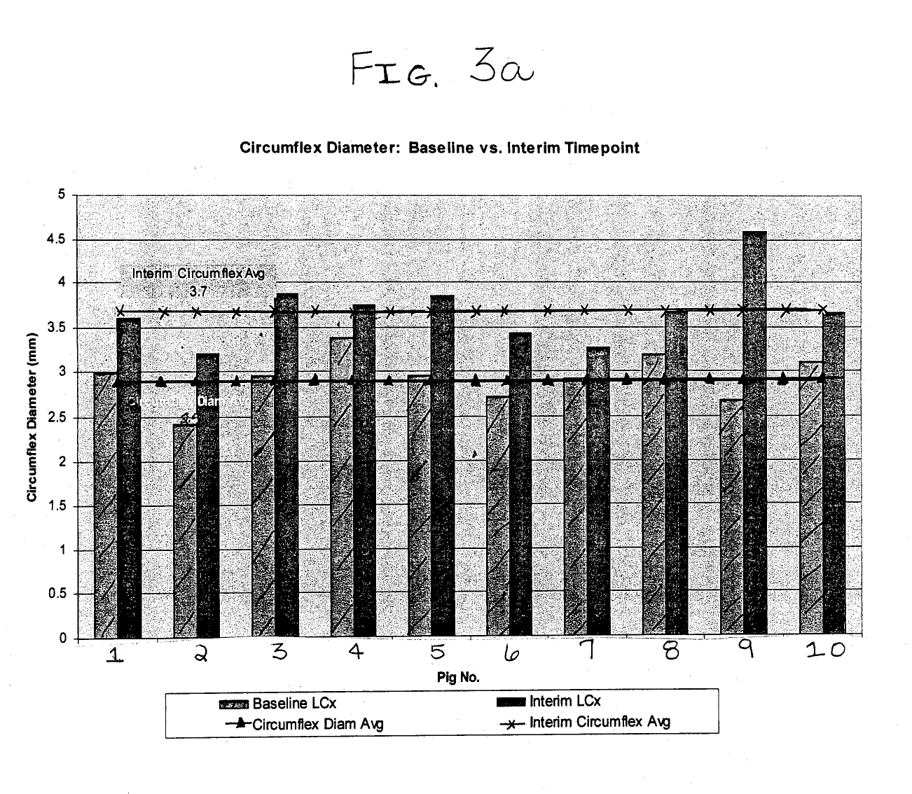

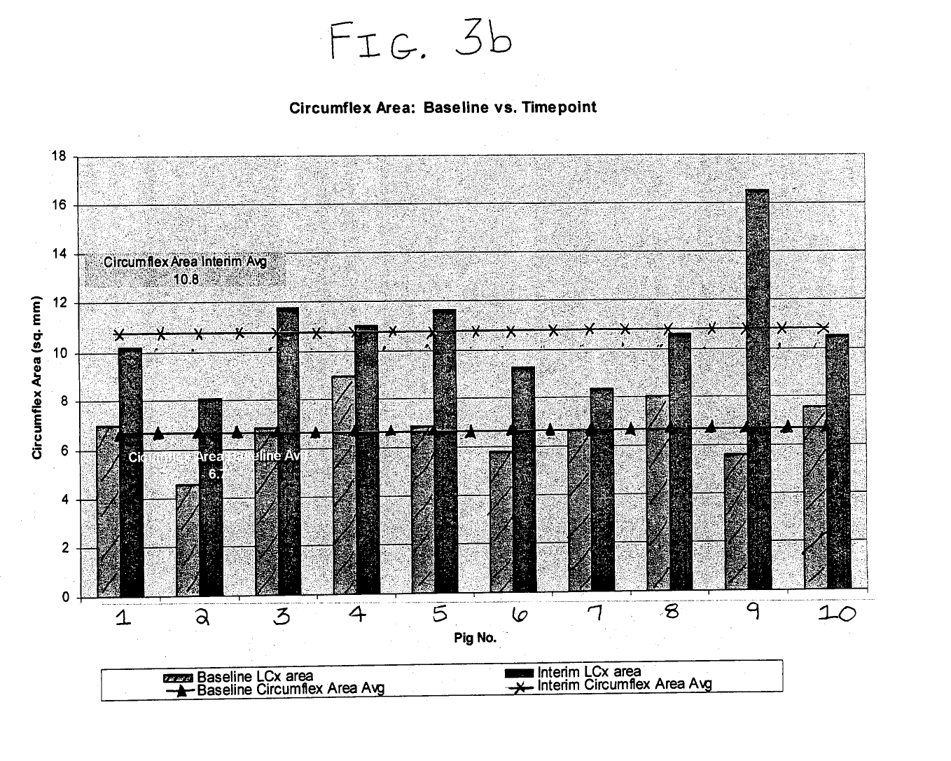

FIG. 3a shows the corresponding change in diameter of the circumflex artery in the pigs of FIGS. 2a and 2b. The diameter measured corresponds to a relatively proximal location of the artery. The average baseline diameter of the circumflex artery, that is the diameter measured prior to placement of the implant in the heart wall between the left ventricle and the left anterior descending artery LAD, was approximately 2.9 mm. On an average of approximately 26.5 days after placing the implant in the pigs, the average circumflex diameter was approximately 3.7 mm, representing an average enlargement of approximately 28%.

FIG. 3b shows a graph containing data regarding the circumflex artery area over time for the same group of ten pigs, and their resultant data, from FIG. 3a. As shown in FIG. 3b, the average baseline area (i.e., prior to placement of the implant in the heart wall) of the circumflex artery, measured at the same location in the artery as the diameter measurement in FIG. 3a, was 6.7 sq. mm. The interim average area of the circumflex measured, on the average, approximately 26.5 days after having placed the implant in the heart wall was 10.8 sq. mm. This represents an average area enlargement of approximately 61%.

Comparing the results shown in FIGS. 2a and 2b with those of FIGS. 3a and 3b shows the increased remodeling effects that the implant has on the vessel in which the implant is placed. That is, the relatively larger increase in diameter and area of the LAD as compared to the circumflex artery may be explained by the greater alteration in flow that the LAD experiences due to is proximity of the implant and the resultant change in flow patterns and shear stresses that occur as a result of the implant.

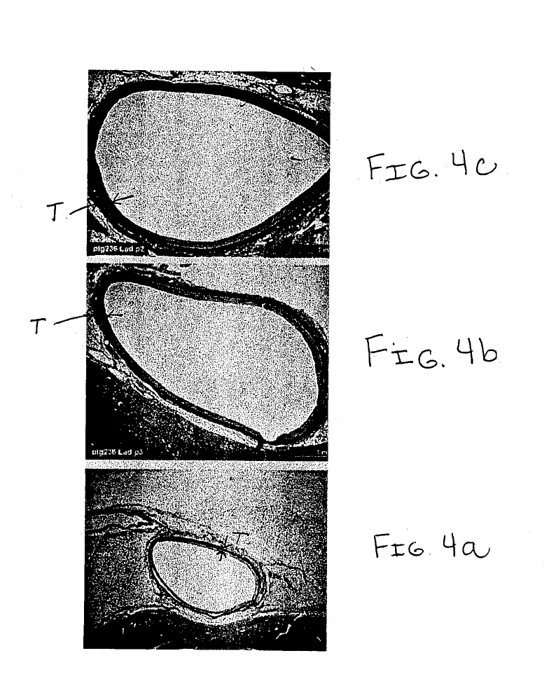

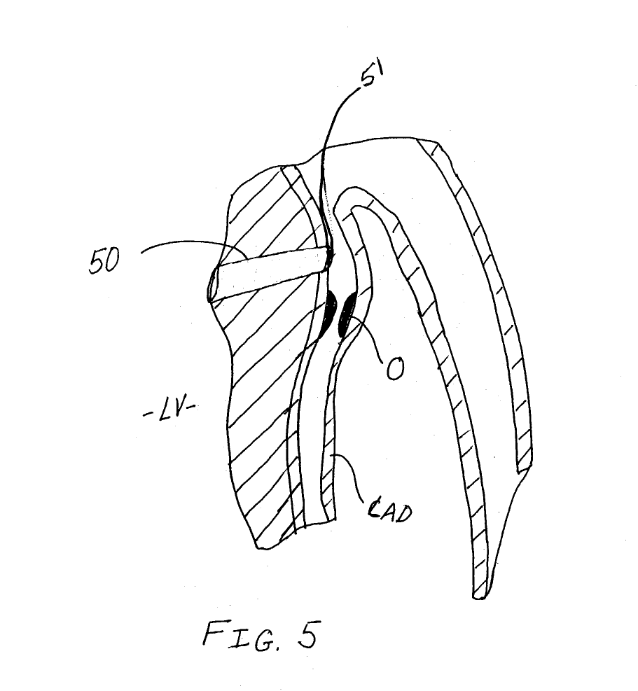

Aside from observing an increase in the proximal diameter and area of the native vessels, a concomitant increase in the thickness of the vessel wall also was observed. FIG. 4a shows the wall thickness T of the circumflex artery, while FIGS. 4b and 4c show the increased wall thickness T of the LAD with its increased size at two different places along the vessel. As these figures show, the increase in diameter of the arteries corresponds to an increase in thickness of the arterial wall. In this example, based on histology and morphometric analysis, the ratio of the increase in diameter to the increase in wall thickness was determined to be approximately 1:1. This increase in wall thickness that occurs when the diameter increases indicates that the enlargement of the arteries is not leading to a hyperplastic or aneurysmal situation. Rather, the remodeling of the vessels that occurs is an adaptive, and not a maladaptive, phenomenon that may preserve the wall hoop stress.

Experiments involving the administration of BrdU also demonstrate that the vessel wall thickness of the LAD increased as the size of the vessel increased due to the flow-dynamics induced vascular remodeling caused by the implant placed in the heart wall between the left ventricle and the LAD. These experiments demonstrated a relatively higher level of ongoing cellular activity (e.g., proliferation) occurring in the proximal LAD (i.e., proximal to the site of the implant) and the distal LAD (i.e., distal to the site of the implant) vessel walls as compared to the vessel walls of the circumflex artery or even more distant arteries. The experiments also showed that the proximal LAD had greater activity than the distal LAD. The experiments using the BrdU further indicate that the type of vessel remodeling occurring as a result of the implant is an adaptive remodeling that causes an increase in wall thickness as well as an overall increase in vessel lumen size, and therefore a preservation in wall hoop stress.

An explanation for the remodeling of the LAD proximal to a ventricular implant, as discussed above and shown in FIGS. 2a-4c, may be that the implant exposes the arterial system to end diastolic left ventricular pressure (EDLVP). The EDLVP is substantially less than the arterial pressure that normally occurs during diastole. By exposing the artery to EDLVP as a result of the ventricular implant, the pressure differential that drives the flow in the artery increases. At the same time, the ventricular implant provides a new path of least resistance during diastole. That is, the resistance to flow of the implant may be lower than the resistance to flow in the distal bed of the LAD. Therefore, some of the diastolic flow goes down the implant (i.e., from the artery through the implant and into the left ventricle). However, not all of the diastolic flow from the proximal artery may go down the implant. Rather, the contemporaneous increase in the pressure differential and the lowering of the pathway resistance, causes the mean flow in the proximal LAD to increase approximately 3 to 5 times, for example, while the diastolic peak flow has been shown to increase by approximately 10 times, for example.

These relatively large increases in flow would be accompanied by increases in luminal wall shear stress in the LAD. Wall shear stress in turn is a significant stimulator of the endothelium, causing it to release vasodilative agents such as nitric oxide and basic fibroblast growth factors, for example. Increased flow through endothelialized vessels therefore may cause positive remodeling of the vessels, for example, an increase in size, as shown in the results of FIGS. 2a-3b.

If a ventricular implant is positioned distal to a stenosis, such as a critical stenosis, for example, the collateral vessels that connect to the distal artery may be influenced by the exposure to increased blood flow via the implant. A similar increase in the driving pressure differential and a decrease in pathway resistance that was realized in the non-stenosed proximal LAD, as described above, may also be at work on the pre-existing collateral vessels connected to the distal LAD. This increase in driving pressure and the decrease in pathway resistance may create a higher diastolic flow through the collaterals, leading to an increased wall shear stress. This in turn could promote positive remodeling of the collateral vessels into conductance vessels. In this manner, the ventricular implant may act as a mechanical arteriogenic stimulator.

Additionally, the ventricular implant also may act as a source of systolic blood flow in the LAD. When the ventricular implant is positioned distal to a total proximal occlusion in an acute pig model, the inventors have found that the implant may provide approximately 50% of the flow that would be provided in the absence of the total occlusion and the implant. In the absence of the ventricular implant, there may be almost no flow in the distal portion of an LAD with a total occlusion acutely in the pig model. The only flow present would be from minor collaterals. Thus, in an acute model with poorly developed collaterals, the ventricular implant can contribute nearly 50% of normal, non-stenosed flow through the LAD.

At a point distal to a total occlusion in an LAD with a ventricular implant positioned distal to the occlusion, the arterial pressure varies between end systolic left ventricular pressure (ESLVP) and EDLVP. As the occlusion is gradually decreased (i.e., as it is altered from 100% blockage to 0% blockage), the diastolic arterial pressure increases from EDLVP (at 100% proximal stenosis) to a diastolic arterial pressure that is just below normal diastolic arterial pressure (at 0% proximal stenosis). The systolic pressure does not vary, however, as a function of the degree of the proximal stenosis. The net effect of this increase in diastolic arterial pressure as the proximal stenosis decreases may be an increase in the mean perfusion pressure distal to the stenosis. In other words, the ventricular implant may become a more effective mechanism to deliver blood flow when the diastolic arterial pressure, and hence the mean perfusion pressure, increases. One way to increase the diastolic pressure in the artery is to valve the ventricular implant flow, i.e., to block the back flow of blood from the artery through the implant and into the left ventricle during diastole. Another way is to decrease the degree of stenosis proximal to the ventricular implant. Yet another way is to increase the degree of collateralization supplying the distal artery.

One way of measuring the success of any arteriogenesis technique is an increase in collateral flow index (CFI), which measures the increase in distal mean pressure as a result of collateral development. As collateral vessels develop into conductance vessels, they contribute greater pressure to the distal vessel, which in turn increases perfusion through the distal bed. The ventricular implant also could stimulate such collateral growth. The growth of collaterals could improve the blood flow delivery of the ventricular implant to at least above the 50% flow discussed above. Thus, the growth of collateral vessels will have two potentially synergistic effects: (1) to increase distal flow through the transformation of collateral vessels from non-conducting vessels to conductance vessels, and (2) to increase the distal diastolic arterial pressure and to thereby increase the efficacy of the ventricular implant.

Prior to applicants' invention, skilled artisans would expect that the exposure of the collateral vessels to EDLVP by the ventricular implant would cause a substantial drop in distal pressure and thus a decrease in flow through the distal collateral bed since the pressure is typically responsible for perfusing the distal bed. That is, skilled artisans would have expected coronary steal through the implant and thus steal of collateral flow as well. However, as explained above, the ventricular implant provides blood flow to the artery during systole. Thus, the mean perfusion pressure and flow provided by the ventricular implant and the collaterals together should be greater than the mean perfusion pressure and flow provided by collaterals alone. In other words, the systolic flow advantages provided by the ventricular implant are greater than the diastolic flow disadvantages associated with the ventricular implant with respect to the perfusion of the distal bed, thereby providing an overall increase in mean perfusion of the distal bed.

Moreover, in the absence of a ventricular implant, as collateral vessels develop and increase distal bed perfusion, the driving pressure differential decreases. Pressure differential dictates flow in the vessel and flow results in shear on the vessel wall. Since shear is the stimulus for collateral development, as the pressure differential decreases further collateral development also may decrease. However, with the flow passage (e.g., ventricular implant) of the present invention, the driving pressure differential leading to the development of collateral vessels may not decrease. This is because the driving pressure differential with a ventricular implant is mean diastolic arterial blood pressure minus EDLVP, as opposed to mean diastolic arterial blood pressure minus intracoronary pressure as measured distal to the occlusion.

Thus, the overall driving pressure with a ventricular implant in place in an occluded artery may be greater than that observed in the absence of the ventricular implant. As an example, in the case of a total coronary artery occlusion, intracoronary pressure distal to the occlusion may be, for example, approximately 30 mmHg to approximately 40 mmHg, and mean diastolic arterial blood pressure may be, for example, approximately 80 mmHg. Thus, the driving pressure in the absence of a ventricular implant in a totally occluded LAD may range from approximately 40 mmHg to approximately 50 mmHg. With the ventricular implant, the driving pressure becomes the mean diastolic arterial blood pressure (e.g., 80 mmHg) minus the EDLVP, which may range, for example, from approximately 10 mmHg to 15 mmHg. Thus, the driving pressure in a totally occluded LAD in the presence of a ventricular implant may range from approximately 65 mmHg to approximately 70 mmHg, for example.

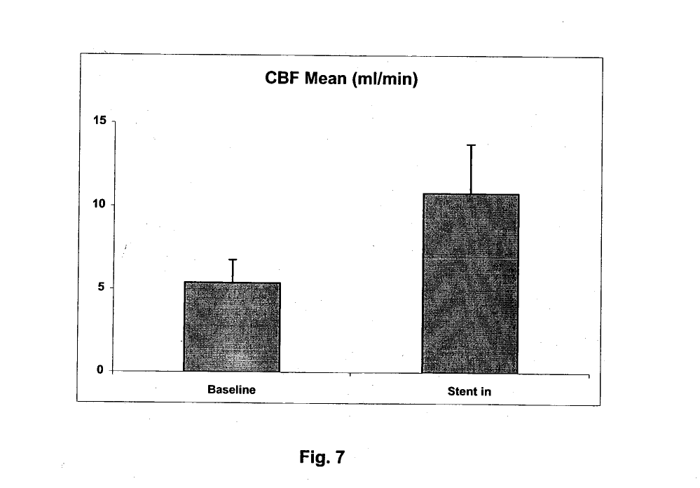

FIG. 7 shows a graph of mean coronary blood flow, as measured by a Transonic flow probe, at a location distal to an occluded vessel in a canine LAD approximately 30 days after implantation of an ameroid occluder. After approximately 30 days, the flow probe placed distal to the ameroid measured an average mean flow of approximately 5 ml/min in three canines. This flow is denoted as the “baseline” flow in the graph of FIG. 7. This “baseline” flow includes the contribution of any collateral vessels that may have developed due to hypoxia induced in the distal bed due to the ameroid occluding the proximal flow.

After making this flow measurement, a ventricular implant, in the form of a stent, was placed in the heart wall between the left ventricle and the LAD at a point in the LAD distal to the ameroid but proximal to the flow probe. In this instance, the average mean flow as measured in three canines increased substantially immediately after implantation to approximately 10 ml/min, as shown by the “Stent in” data of FIG. 7. Thus, contrary to what skilled artisans would have expected prior to the inventors' discovery, the ventricular implant did not steal coronary flow. That is, based on the increase in flow that was measured after placing the stent in the heart wall, it is apparent that the “baseline” flow is not being diverted into the stent such that the overall flow through the LAD decreases from the “baseline” measured flow.

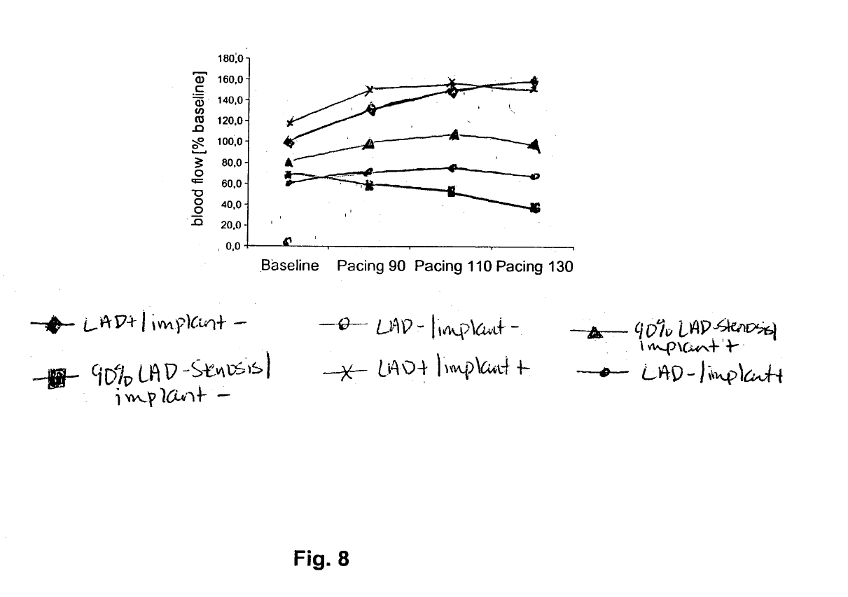

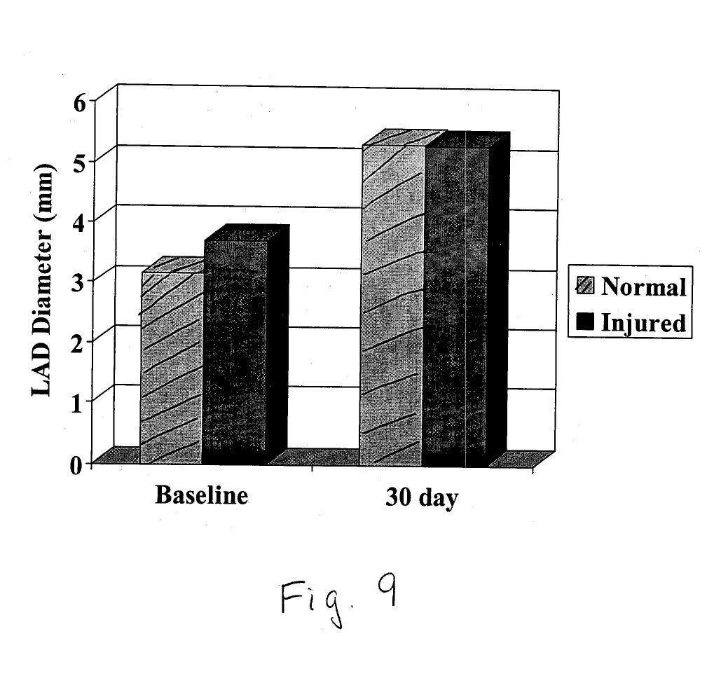

In an attempt to understand the effect of the ventricular implant on coronary flow in a coronary artery having a high-grade stenosis (i.e., 90% occluded), and specifically to understand whether the ventricular implant would steal coronary flow as those skilled in the art would predict prior to this invention, the inventors performed further experiments. One of the experiments included determining how a ventricular implant affects coronary reserve when positioned in the heart wall of several pigs distal to a high grade stenosis. FIG. 8 shows the results of this experiment.

To begin with, the flow in the LAD, without any stenosis and without an implant placed in the heart wall, was measured and normalized to 100%. The heart was then paced, and as the demand for bloodflow increased, the flow increased to meet the demand. This condition is represented in FIG. 8 by the line “LAD+/implant−”. Next, the LAD was fully occluded (i.e., 100% stenosis) by ligating the vessel, resulting in a 0% distal flow through the LAD. This condition is represented in FIG. 8 by the “LAD−/implant−” data point. The ligation was removed, and a 90% stenosis was created. This 90% stenosis was created acutely by inflating a balloon within the vessel so as to partially occlude the vessel. The result was a decrease in distal flow by 30% of the “baseline” flow (i.e., a 30% decrease of the 100% normalized flow from the “LAD+/implant−” situation). The heart was then paced. Under pacing, the flow through the LAD was unable to meet the increase in demand, as indicated by the drop in flow as a percent of baseline. The “90%−LAD Stenosis/implant−” line in FIG. 8 shows this condition.

Next, the 90% stenosis was removed and the ventricular implant was placed in the heart wall between the left ventricle and the LAD. The heart was then paced and the flow measured, as shown in FIG. 8 by the “LAD+/implant+” line. In this case, the flow was slightly higher than the 100% normalized “baseline” measured previously and was able to meet the increased demand resulting from pacing of the heart. A 90% stenosis was then created and the ventricular implant was left in place. The flow distal to the ventricular implant was approximately 20% less than the flow measured for the “LAD+/implant+” condition, but was slightly greater than the 90% stenosis alone with no implant. When the heart was paced, the flow increased to meet the demand, as shown by the line “90% LAD−Stenosis/implant+” in FIG. 8. In the final case, the proximal LAD was completely ligated. The flow produced solely from the ventricular implant was approximately 60% of native LAD flow. During pacing, the flow increased in response to an increase in demand. The flow for this condition is shown by the “LAD−/implant+” line in FIG. 8.

From these experiments, the inventors have discovered that, at rest, in the presence of a high-grade stenosis, the flow distal to the ventricular implant is higher than the flow when the ventricular implant is absent. Thus, the ventricular implant does not steal flow at rest, as skilled artisans prior to the invention would have thought. Under pacing conditions, the flow in the presence of a 90% stenosis alone is unable to meet the increased demand. However, when the ventricular implant is placed distal to the high-grade stenosis (e.g., 90%), the flow increases as the demand increases. The flow at rest and under pacing was approximately 20% higher in the case of the ventricular implant positioned distal to a 90% stenosis in the LAD when compared to the ventricular implant positioned distal to a total occlusion in the LAD. An explanation for this may be that the preservation of diastolic arterial pressure that occurs with a 90% stenosis improves the blood flow through the ventricular implant into the LAD.

Overall, the data of FIG. 8 suggest that in the presence of a total occlusion and a collateralized distal bed or in the presence of a high-grade stenosis, which may or may not be a total stenosis, the systolic contribution of the ventricular implant may augment, rather than decrease, the blood flow distal to the implant.

Aside from providing vascular remodeling responses in noninjured vasculature, the inventors have discovered that similar flow-dynamics-induced vascular remodeling also occurs in the setting of diseased, injured vessels, such as those associated with diffusely stenosed vessels, for example, as may be found in diabetics or individuals with high cholesterol. An experiment using 8 pigs was conducted in which an unoccluded proximal LAD was subject to balloon injury. Thirty days after the balloon injury to the proximal LAD, a ventricular implant in the form of a stent was placed in the heart wall between the left ventricle and the LAD at a point distal to the balloon injury. Various angiographic, perfusion, pressure, and histomorphometric data were collected.

The angiographic data demonstrated that the remodeling of the coronary vessel that occurred was similar to that observed in an uninjured LAD, as described above. Further, the histomorphometric data demonstrated that the remodeling was adaptive, i.e., as the vessel diameter grew the wall thickness increased.

Some exemplary results taken from the study of the balloon injured vessel are presented in FIG. 9. FIG. 9 contains averaged data from two groups of animals showing the diameter of the LAD in balloon injured and noninjured vessels, each with a stent placed in the heart wall. The diameter data shown were measured proximal to the site of the implant in the LAD. The baseline data represents measurements taken at the time when the implant was installed in the heart wall. The baseline measurements show that the LAD diameter in the injured vessel group was slightly greater than the LAD in the noninjured group. Thirty days after implanting the stent in the heart wall, the data in FIG. 9 shows that the balloon-injured LAD diameter and the noninjured LAD diameter are approximately the same and have both increased to approximately 5 mm. Thus, the results of FIG. 9 show that the balloon-injured vessel also exhibits flow-dynamics-induced vascular remodeling effects in the form of enlargement of the proximal LAD similar to those of the noninjured vessel.

The inventors have observed an unexpected alteration of the flow dynamics associated with the formation of a blood flow passage (e.g., as formed by an implant, such as a stent, for example) in the heart wall. For example, the overall flow through the coronary artery, including mean flow in the proximal LAD and diastolic peak flow, has been observed to increase. Moreover, the inventors have observed remodeling effects in native blood vessels, for example, an increase in the size of those vessels, including both the vessel diameters and the thickness of the vessel wall. Further, the growing of collateral vessels also has been observed. The inventors also have found that the adaptive, positive vascular remodeling effects from the flow-dynamics-induced remodeling occur in both injured as well as noninjured vessels.

Based on these findings, i.e., the significant flow-dynamics-induced vascular remodeling, the inventors also have discovered a treatment for certain types of patients having occluded and/or diseased vessels that previously would have been either very difficult or impossible to treat. That is, in patients in which conventional coronary vessel procedures are difficult due to, for example, relatively narrow, noncompliant vessels, such as in diabetic patients and the like, the placement of an implant (or other formation of a blood flow passage) as described above may cause flow-dynamics-induced remodeling of the vasculature so as to permit further coronary vessel procedures to subsequently be performed. The enlargement of the once restricted and relatively rigid artery may provide enough room for anastomosis of a bypass graft, insertion of a balloon catheter, placement of an intracoronary stent, performance of an atherectomy, etc.

For example, the overall enlargement of the vessel may increase the number of viable sites at which a surgeon may attach the graft. Also, the enlargement of the vessel diameter may facilitate the maneuverability of catheters and other similar tools used in various other conventional coronary vessel procedures, such as balloon angioplasty, intracoronary stenting, and the like. For instance, a balloon catheter that may have been hindered from free advancement through the relatively restricted or rigid vessel, may become more easily navigable within the vessel due to the vascular remodeling, and in particular the enlargement, that takes place in conjunction with the direct blood flow from a heart chamber to the vessel through the passage formed in the heart wall. Similarly, placement of an intracoronary stent may be difficult in a relatively narrow, rigid artery. The enlargement of the artery resulting from the flow-dynamics-induced vascular remodeling may permit the expansion of and placement of the intracoronary stent within the diseased artery.

In an aspect of the invention, a ventricular implant 10 (which may be in the form of a stent or conduit, for example) may be placed in the heart wall, as shown in FIG. 1, followed by an anastomosis of a bypass graft (not shown) once a desired enlargement of the artery has taken place. The implant 10 may be permanently implanted so as to remain as an adjunctive treatment with the bypass graft or other conventional coronary vessel procedure, as will be discussed in more detail shortly. Alternatively, the implant may function temporarily to permit blood flow therethrough. For example, the implant may be temporarily implanted, for example it may be removed prior to or at the time of implanting the bypass graft, or the implant may be biodegradable or bioabsorbable. The implant may resorb after a period of time has elapsed, such as a predetermined time of 30 days or 90 days, or any other time period sufficient to permit enlargement of the artery to a desired degree. Alternatively, the implant may be permitted to resorb after implantation of the bypass graft or performance of other conventional coronary vessel procedure.

In exemplary embodiments of the invention, the formation of the passage in the heart wall and the corresponding vascular remodeling is useful as a staging technique to first enlarge the vessel so as to permit the later performance of a conventional coronary vessel procedure on the enlarged, remodeled vessel. Additionally, forming the passage in the heart wall between the ventricle and the artery also may be useful as an adjunctive technique. That is, the positive remodeling effects associated with the formation of the passage in the heart wall to permit direct blood flow between the ventricle and the artery may improve the patency of the coronary vessel associated with conventional coronary vessel procedures. For example, the flow-dynamics-induced remodeling may counterbalance negative effects, including restenosis, often associated with conventional coronary vessel procedures, such as balloon angioplasty.

An example of utilizing the formation of the passage as an adjunctive technique includes forming the passage in the heart wall substantially contemporaneously with the performance of a balloon angioplasty procedure on a diseased artery. Once the balloon angioplasty procedure has been completed, the passage could be maintained patent for a relatively long time period or even permanently so as to counteract, via the flow-dynamics-induced vascular remodeling, the tendency of the vessel to restenose after angioplasty has been performed. Similar improvements may be obtained with regard to the patency of a bypass graft by using the formation of the heart wall passage and flow-dynamics-induced vascular remodeling phenomenon as an adjunctive technique with CABG.

In another exemplary embodiment, a passage may be formed in the heart wall of a transplanted heart so as to achieve flow-dynamics-induced vascular remodeling. In this way, the negative remodeling effects often associated with heart transplants, such as late narrowing and loss of branches of the coronary tree, for example, may be counteracted. By providing the flow-dynamics-induced vascular remodeling effects described herein due to the formation of the passage in the heart wall of the donor heart, the risk of accelerated coronary artery disease may be diminished.

In yet another exemplary embodiment, the passage in the heart wall may be formed so as to provide blood flow between a heart chamber and diseased vessel, and the consequent remodeling of the vasculature may be monitored and assessed so as to select a subsequent therapy based on the remodeling effects that occur. By way of example, the enlargement of a coronary artery may be monitored after the placement of an implant in a heart wall between the left ventricle and the artery. Based on the observed enlargement, a subsequent therapy may be selected to further treat the patient having the diseased artery. For example, a particular anastomosis site may be selected for the bypass graft based on the observed enlargement of the artery at particular locations. In another exemplary aspect, after the monitoring of the enlargement, the type of further therapy may be selected based on the amount of enlargement observed. For example, a particular size balloon or a particular size coronary stent may be selected.

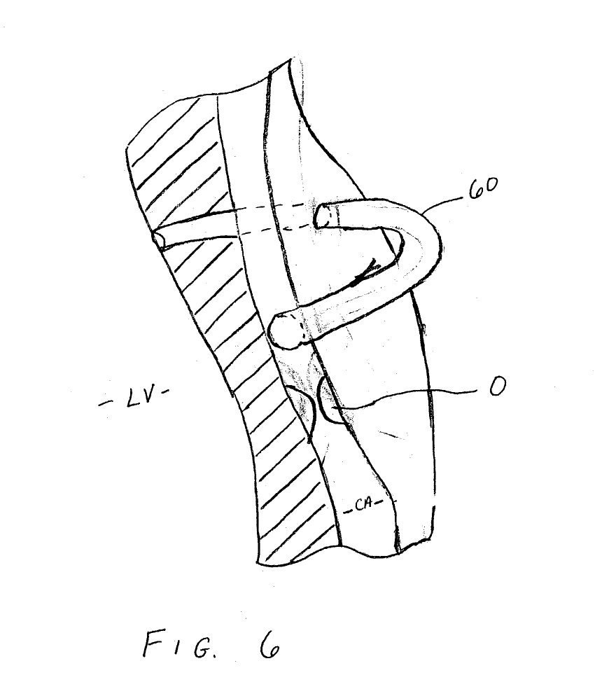

In another exemplary embodiment according to an aspect of the invention, a passage is created in the heart wall between a heart chamber and an occluded vessel so as to provide a direct blood flow path between the heart chamber and a point in the vessel upstream (i.e. proximal) of an occlusion. FIG. 5 illustrates an implant 50 in the heart wall so as to provide a direct blood flow passage between the left ventricle LV and a left anterior descending artery LAD. The implant 50 is placed so that the end 51 of the implant in communication with the LAD is disposed upstream of the stenotic lesion O. In a manner similar to that described above, the implant (which may be in the form of a collapsible stent, for example) placed in this manner may induce relatively high flow rates into the LAD and otherwise alter flow dynamics within the LAD. These various changes in flow dynamics will in turn induce remodeling of the LAD and other vasculature, including enlargement of the arterial diameter substantially at the location of the occlusion and possibly along other regions as well. In essence, the formation of the passage in the heart wall so as to provide blood flow between the heart chamber and a location upstream of an occluded coronary vessel, along with the consequent remodeling of the vasculature, causes a sort of “biological angioplasty” resulting in the increased perfusion of the myocardium.

This “biological angioplasty” approach may be used alone as a sole therapy, without the need for conventional coronary vessel procedures. Alternatively, the approach could be combined with other traditional medical therapies, such as drug treatments to lower cholesterol, or the like. Further, as discussed above, the implant also may function only temporarily. For example, the implant may be only temporarily placed in the heart wall until a desired enlargement of the artery occurs or, alternatively, the implant may be permanently placed in the heart wall.

FIGS. 1 and 5 show the formation of a passage (via placement of an implant 10, 50) in the heart wall positioned such that one end portion of the passage has an opening in flow communication with the left ventricle LV and the other end portion extends through a posterior wall of the left anterior descending artery LAD and has an opening in flow communication with the lumen of the artery. As an alternative, a passage connecting the left ventricle LV and the artery may have one end portion with an opening in flow communication with the left ventricle LV and another end portion extending through an anterior wall of the artery and having an opening in flow communication with the lumen of the artery. In this case, the portion of the passage between the two end portions would extend through the heart wall and into the pericardial space. FIG. 6 shows an exemplary embodiment of a passage 60 formed in this manner, wherein the opening in flow communication with the lumen of the artery is positioned proximal to an occlusion O. It should be noted that it is within the scope to place the opening distal to the occlusion. However, because the artery may be relatively narrower and more rigid distal to the artery, this technique may be easier to implement when placing the opening proximal to the occlusion, as shown in FIG. 6.

In any of the techniques discussed above, catheter-based delivery methods may be used to deliver the implant or otherwise form the passage. For example, the implant may be delivered, or the passage may be otherwise formed, percutaneously in a manner similar to that disclosed in U.S. Pat. No. 5,429,144. Alternatively, conventional surgical approaches for delivery of the implant or otherwise forming the passage may be utilized.

As mentioned above, depending on the situation in which the formation of the passage to effect flow-dynamics-induced vascular remodeling will be used, it may be desirable to form a passage that remains open (patent) for a time period and then occludes, or otherwise ceases to function (e.g., closes off), after the desired effect of the corresponding remodeling has been accomplished. This time period may be as short as one month, for example when used as a staging procedure, such as to subsequently implant a bypass graft or intracoronary stent. Alternatively, it may be as long as a year or more, for example when used as an adjunctive procedure, such as in conjunction with the performance of balloon angioplasty.

Such a time controlled passage could be achieved in numerous ways. One way to achieve this time controlled passage includes providing a coated implant. The coating may be configured so as to prevent thrombus formation and/or tissue ingrowth for a time period. Once this time period has passed, the coating may degrade so as to either initiate a closing response or to permit thrombus formation and tissue ingrowth. Another way to achieve this time controlled passage may be to provide an implant that degrades after a time period. Upon degradation of the implant, the passage through the heart wall between the heart chamber and the vessel would be allowed to close.

In an alternative, again depending on the application for which the formation of the passage and flow-dynamics-induced vascular remodeling will be used, it may be desirable to maintain the passage patent so as to affect long-term control over the remodeling effects. Such long term patency of the passage may be achieved, for example, by coating of the passage itself with tissue ingrowth and thrombus preventing substances or by providing an implant with such a coating.

In another exemplary embodiment, substances such as pharmacological agents, drugs, genes, and other similar suitable substances, may be delivered in conjunction with the formation of the passage. These substances could include agents that accelerate and/or increase the vascular remodeling effects associated with the passage or other flow dynamics altering mechanical device. Such pharmacological agents may include, for example, arteriogenic agents and the like. Examples of such agents include vascular endothelial-derived growth factor (VEGF) and fibroblast growth factor (FGF). The substances could be delivered via a coating, covering, or a three-dimensional matrix on an implant placed so as to form the passage, or via other suitable delivery means.

It will be apparent to those skilled in the art that various modifications and variations can be made in the devices and related methods for inducing vascular remodeling via an alteration in flow dynamics and for treating diseased vascular systems via such remodeling without departing from the scope or spirit of the invention. For example, the particular patency time periods described above may be modified as desired. Moreover, although reference has been made in the exemplary embodiments to treating a diseased coronary artery and to the blood flow dynamics relating to the left ventricle and coronary artery, it is envisioned that other vascular structures may be treated and other connections between various vascular structures may be made using the devices and methods of the present invention. For example, those skilled in the art would understand that flow passages could be formed between two chambers, between a chamber and a vein, between two vessels, between an artery and a vein, and between virtually any other vascular structures. Those having skill in the art also would recognize how the devices and methods could be employed and/or modified to remodel other vascular structures and to treat other diseased portions of the vascular system, such as coronary veins, for example, and other non-coronary vasculature. In addition, skilled artisans would recognize that to treat a diseased coronary artery, connections between the artery and vascular structures other than the left ventricle also may be employed. For example, connections to the right ventricle, right atrium, or left atrium may be made.

It should be understood that the invention is not limited to the examples discussed in the specification. Rather, the present invention is intended to cover modifications and variations.

What is claimed is:

1. A method of treating a diseased coronary vessel, the method comprising:
providing a blood flow passage between a heart chamber and the coronary vessel such that enlargement of at least a portion of the coronary vessel occurs; and
selecting a further therapy to treat the diseased vessel based on the enlargement of the vessel.

2. The method of claim 1, further comprising assessing the enlargement of the vessel.

3. The method of claim 1, wherein selecting the further therapy includes selecting a further therapy chosen from bypass grafting, angioplasty, atherectomy, and intracoronary stenting.

4. The method of claim 1, wherein providing a blood flow passage includes providing a blood flow passage that functions temporarily.

5. The method of claim 1, wherein providing a blood flow passage includes placing an implant in a heart wall surrounding the chamber.

6. The method of claim 5, wherein placing the implant includes placing a conduit in the heart wall.

7. The method of claim 1, wherein providing the blood flow passage includes providing a blood flow passage having an opening in flow communication with the vessel at a point proximal to an occlusion in the vessel.

8. The method of claim 1, wherein providing the blood flow passage includes providing a blood flow passage having an opening in flow communication with the vessel at a point distal to an occlusion in the vessel.

9. The method of claim 1, wherein providing the blood flow passage includes providing a blood flow passage that provides direct flow communication between the heart chamber and the coronary vessel.

10. A method of treating a coronary vessel having an occlusion, the method comprising:
implanting a device in a heart wall so as to cause blood to flow through the device into the vessel at a point proximal to the occlusion.

11. The method of claim 10, wherein implanting the device includes implanting a conduit.

12. The method of claim 11, wherein implanting the conduit includes implanting the conduit in a heart wall between a heart chamber and the coronary vessel.

13. The method of claim 12, wherein implanting the conduit further includes implanting an end portion of the conduit proximate an anterior wall of the coronary vessel.

14. The method of claim 11, wherein implanting the conduit includes implanting the conduit in a heart wall so that an end portion of the conduit is proximate a posterior wall of the coronary vessel.

15. The method of claim 10, further comprising delivering at least one substance chosen from pharmacologic agents, genes, and drugs proximate the device.

16. A method of treating a heart, the method comprising:
placing an implant having a passage in a heart wall between a heart chamber and a coronary vessel so as to permit blood flow through the passage between the chamber and the coronary vessel during at least a portion of a cardiac cycle and induce vascular remodeling; and
after a predetermined time period, ceasing to permit the blood flow through the passage throughout the entire cardiac cycle.

17. The method of claim 16, wherein the implant includes a coating that degrades after a period of time.

18. The method of claim 17, wherein the coating prevents thrombus formation.

19. The method of claim 17, wherein the coating prevents tissue ingrowth.

20. The method of claim 16, wherein the implant degrades after a period of time.

21. The method of claim 16, further comprising providing an implant that degrades after the predetermined time period.

22. The method of claim 16, further comprising removing the implant from the heart wall after the predetermined time period.

23. The method of claim 16, wherein inducing vascular remodeling includes increasing the diameter of at least a portion of the coronary vessel.

24. The method of claim 16, wherein inducing vascular remodeling includes inducing arteriogenesis in coronary vasculature.

25. The method of claim 16, wherein the implant remains in the heart wall temporarily.

26. The method of claim 16, wherein the predetermined time period is a time period at least sufficient to cause desired vascular remodeling.

27. A method of treating a heart, the method comprising:
providing a blood flow passage between a heart chamber and a coronary vessel so as to permit blood to flow between the heart chamber and the coronary vessel and induce vascular remodeling; and
delivering at least one substance chosen from pharmacologic agents, genes, and drugs proximate the blood flow passage to accelerate the vascular remodeling.

28. The method of claim 27, wherein inducing vascular remodeling includes increasing the diameter of at least a portion of the coronary vessel.

29. The method of claim 27, wherein inducing vascular remodeling includes inducing arteriogenesis in coronary vasculature.

30. The method of claim 27, wherein providing a blood flow passage includes implanting a conduit in a heart wall surrounding the chamber.

31. The method of claim 27, wherein the conduit includes the at least one substance.

32. The method of claim 27, wherein providing the blood flow passage includes providing a blood flow passage between a heart chamber and a coronary vessel so as to permit blood to flow directly between the heart chamber and the coronary vessel.

33. A method of treating a heart having an injured coronary vessel, the method comprising:
providing a blood flow passage between a heart chamber and an injured coronary vessel so as to permit blood to flow between the heart chamber and the injured coronary vessel,
wherein providing the blood flow passage induces vascular remodeling of the heart.

34. The method of claim 33, wherein the injured coronary vessel includes a coronary artery.

35. The method of claim 33, wherein inducing vascular remodeling includes increasing the diameter of at least a portion of the injured coronary vessel.

36. The method of claim 33, wherein inducing vascular remodeling includes inducing arteriogenesis in coronary vasculature.

37. The method of claim 33, wherein providing a blood flow passage includes implanting a conduit in a heart wall surrounding the chamber.

38. The method of claim 33, wherein providing the blood flow passage includes providing a blood flow passage between a heart chamber and a coronary vessel so as to permit blood to flow directly between the heart chamber and the coronary vessel.

39. A method of treating a heart, the method comprising:
providing a blood flow passage between a heart chamber and a coronary vessel so as to permit blood to flow between the heart chamber and the coronary vessel to induce vascular remodeling; and
providing a further vascular therapy for treating the heart after the providing of the blood flow passage.

40. The method of claim 39, wherein providing the further vascular therapy includes providing a myocardial growth factor therapy.

41. The method of claim 39, wherein providing the further vascular therapy includes providing a therapy chosen from bypass grafting, angioplasty, atherectomy, and intracoronary stenting.

42. The method of claim 39, wherein inducing vascular remodeling includes increasing the diameter of at least a portion of the coronary vessel.

43. The method of claim 42, wherein the portion of the coronary vessel includes a site to receive a bypass graft.

44. The method of claim 39, wherein inducing vascular remodeling includes inducing arteriogenesis in coronary vasculature.

45. The method of claim 39, wherein providing a blood flow passage includes implanting a conduit in a heart wall surrounding the chamber.

46. The method of claim 39, wherein providing the blood flow passage includes providing a blood flow passage between a heart chamber and a coronary vessel so as to permit blood to flow directly between the heart chamber and the coronary vessel.

47. The method of claim 39, further comprising assessing the vascular remodeling and selecting the further vascular therapy based on the assessing step.

48. A method of performing a heart transplant, the method comprising:
providing a blood flow passage between a heart chamber and a coronary vessel of a transplant heart; and
performing the heart transplant by implanting the transplant heart.

49. The method of claim 48, wherein providing the blood flow passage induces vascular remodeling.
